(19) United States
(12) Patent Application Publication
(10) Pub. No.: US 2022/0133525 A1
(43) Pub. Date: May 5, 2022

(54) MENSTRUAL CUP

(71) Applicant: Clean Light Laboratories LLC, Scottsdale, AZ (US)

(72) Inventor: Carrie Martz, Scottsdale, AZ (US)

(21) Appl. No.: 17/518,357

(22) Filed: Nov. 3, 2021

Related U.S. Application Data
(60) Provisional application No. 63/109,023, filed on Nov. 3, 2020.

(51) Int. Cl. A61F 5/455

(52) U.S. Cl. CPC A61F 5/455

(57) ABSTRACT

A menstrual cup having improved leakage protection along with enhanced features for facilitating its swift and easy removal from a user that includes a cup shaped main body having an open top end and a closed bottom end, an elongated loop member attached to an exterior of the closed bottom of the main body, a lip member positioned about an outer circumference of the open top of the cup shaped main body, and one or more of a spiral ribbing located about an interior surface of the cup shaped main body extending from the open top of the main body to the closed bottom of the main body, a plurality of openings extending through the cup shaped main body located near the open top of the main body and spaced equidistantly from one another about a circumference of the main body, and a plurality of raised oblong shaped rings equidistantly spaced apart from one another on an exterior surface of the cup shaped main body near the closed bottom of the main body which form suction release indentations.

### CROSS-REFERENCE TO RELATED APPLICATIONS

This patent application claims priority to provisional patent application having Ser. No. 63/109,023, filed Nov. 3, 2020, which is herein incorporated by reference in its entirety.

### FIELD OF INVENTION

The present invention is directed to a reusable menstrual cup having improved features to prevent the cup from leaking and to enable easy removal of the cup.

### BACKGROUND OF THE INVENTION

Reusable menstrual cups have been known and in use for some time. However, many menstrual cups do not adequately protect from leakage around the rim of the cup and many are difficult to remove when it is time for a user to remove the cup.

### SUMMARY OF THE INVENTION

The present invention is directed to a new and improved menstrual cup having an elongated loop positioned at a bottom of the cup and suction release indentations positioned about a circumference of the cup near a bottom of the cup to enable easy removal of the cup by a user. The menstrual cup of the present invention also includes interior spiral ribbing in a helix of corkscrew like configuration to facilitate “pop out” removal of the cup while enabling the cup to retain its shape while being used inside of a user's body. The menstrual cup of the present invention also includes a lip member positioned about a top outer circumference of the cup to enable the creation of a better seal with a user's cervix when the cup is in use and to prevent leaking of menstrual contents contained in the cup when in use. The menstrual cup of the present invention also includes a plurality of openings extending through the cup and spaced equidistantly apart from one another near the top of the cup about a circumference of the cup to assist in easy removal of the cup by assisting in releasing pressure/suction when the suction release indentations on the menstrual cup are pressed/compressed by a user's fingers when removing the cup from the inside of a user's body.

In one exemplary embodiment, the menstrual cup of the present invention comprises a cup shaped main body having an open top and a closed bottom, a spiral ribbing located about an interior surface of the cup shaped main body extending from the open top of the main body to the closed bottom of the main body, a plurality of openings extending through the cup shaped main body located near the open top of the main body and spaced equidistantly from one another about a circumference of the main body, and an elongated loop member attached to an exterior of the closed bottom of the main body. The menstrual cup may also include a lip member positioned about an outer circumference of the open top of the cup shaped main body. The menstrual cup may also include a plurality of raised oblong shaped rings equidistantly spaced apart from one another on an exterior surface of the cup shaped main body near the closed bottom of the main body and a plurality of raised ridge members positioned on the exterior surface of the cup shaped main body such that each raised ridge member connects to two of the raised oblong shaped rings (e.g. opposite ends of each raised ridge member are connect to a different raised oblong shaped ring so that the plurality of raised ridge members end up connecting all of the raised oblong shaped rings to one another). The menstrual cup may also include a first contiguous ridge member that extends about an outer circumference of the cup shaped body on the exterior of the main body between the plurality of raised ridge members and the closed bottom of the main body. The menstrual cup may further include a second continuous ridge member that extends about an outer circumference of the cup shaped body on the exterior of the main body between the first continuous ridge member and the closed bottom of the main body such that it touches opposing sides of the elongated loop member. The plurality of openings extending through the cup shaped main body may include four openings and the plurality of raised oblong shaped rings may include four raised oblong shaped rings. Although the plurality of openings are spaced about a circumference of the cup shaped main body near the open top of the main body and the plurality of raised oblong shaped rings are positioned about a circumference on the exterior of the cup shaped main body near the closed bottom of the main body, the plurality of openings are spaced such that they are positioned between, and not directly above, the plurality of raised oblong shaped rings.

In another exemplary embodiment of the menstrual cup of the present invention, the menstrual cup includes a cup shaped main body, a plurality of raised oblong shaped rings equidistantly spaced apart from one another on an exterior surface of the cup shaped main body near the closed bottom of the main body, a plurality of raised exterior ridge members positioned on the exterior surface of the cup shaped main body such that each raised exterior ridge member connects to two of the raised oblong shaped rings thereby connecting all of the raised oblong shaped rings to one another, and an elongated loop member attached to an exterior of the closed bottom of the main body. The menstrual cup may also include a first continuous ridge member that extends about an outer circumference of the cup shaped body on the exterior of the main body between the plurality of raised ridge members and the closed bottom of the main body and a second continuous ridge member that extends about an outer circumference of the cup shaped body on the exterior of the main body between the first continuous ridge member and the closed bottom of the main body such that it touches opposing sides of the elongated loop member. The menstrual cup may further include a lip member positioned about an outer circumference of the open top of the cup shaped main body, a spiral ribbing located about an interior surface of the cup shaped main body extending from the open top of the man body to the closed bottom of the main body, and/or a plurality of openings extending through the cup shaped main body located near the open top of the main body and spaced equidistantly from one another about a circumference of the main body. The plurality of openings extending through the cup shaped main body may include four openings and the plurality of raised oblong shaped rings may include four raised oblong shaped rings. Although the plurality of openings are spaced about a circumference of the cup shaped main body near the open top of the main body and the plurality of raised oblong shaped rings are positioned about a circumference on the exterior of the cup shaped main body near the closed bottom of the main body, the plurality of openings are spaced such that they are positioned between, and not directly above, the plurality of raised oblong shaped rings.

In yet another exemplary embodiment, the menstrual cup of the present invention includes a cup shaped main body having an open top and a closed bottom, a spiral ribbing located about an exterior surface of the cup shaped main body extending from the open top of the main body to the closed bottom of the main body, a plurality of openings extending through the cup shaped main body located near the open top of the main body and spaced equidistantly from one another about a circumference of the main body, a plurality of raised oblong shaped rings equidistantly spaced apart from one another on an exterior surface of the cup shaped main body near the closed bottom of the main body, and an elongated loop member attached to an exterior of the closed bottom of the main body. The plurality of openings extending through the cup shaped main body may include four openings and the plurality of raised oblong shaped rings may include four raised oblong shaped rings. Although the plurality of openings are spaced about a circumference of the cup shaped main body near the open top of the main body and the plurality of raised oblong shaped rings are positioned about a circumference on the exterior of the cup shaped main body near the closed bottom of the main body, the plurality of openings are spaced such that they are positioned between, and not directly above, the plurality of raised oblong shaped rings. The cup shaped main body, the spiral ribbing, the plurality of openings, the plurality of raised oblong shaped rings, and the elongated loop member may all be formed from one molded piece.

Further, all of the elements contained in each of the above described first and second exemplary embodiments of the menstrual cup of the present invention may also be formed form one molded piece to ensure strength and durability of the menstrual cup.

### BRIEF DESCRIPTION OF THE DRAWINGS

The foregoing and other features of the present disclosure will become more fully apparent from the following brief description, taken in conjunction with the accompanying drawings and reference numbers. Understanding that these drawings depict only some embodiments in accordance with the disclosure and are, therefore, not to be considered limiting of its scope, the disclosure will be described with additional specificity and detail through use of the accompanying drawings.

FIG. 1 is a perspective view of an exemplary embodiment of the menstrual cup of the present invention;

FIG. 2 is a front elevational view of the exemplary embodiment of the menstrual cup shown in FIG. 1;

FIG. 3 is a side elevational view of the exemplary embodiment of the menstrual cup shown in FIG. 1;

FIG. 4 is a bottom plan view of the exemplary embodiment of the menstrual cup shown in FIG. 1;

FIG. 5 is a top plan view of the interior of the exemplary embodiment of the menstrual cup shown in FIG. 1;

FIG. 6 is a front view showing dimensions for one size of the exemplary embodiment of the menstrual cup shown in FIG. 1;

FIG. 7 is a top plan view showing the interior of the menstrual cup shown in FIG. 6;

FIG. 8 is a front view showing dimensions for another size of the exemplary embodiment of the menstrual cup shown in FIG. 1; and

FIG. 9 is a top plan view showing the interior of the menstrual cup shown in FIG. 8.

### DETAILED DESCRIPTION OF EXEMPLARY EMBODIMENTS

The following reference numbers refer to the reference numbers contained in the above drawings and include key features of the menstrual cup of the present invention:
10 menstrual cup
12 cup shaped main body of menstrual cup
14 open top of menstrual cup
16 closed bottom of menstrual cup
17 opening in elongated loop member
18 elongated loop member
19 raised oblong shaped rings
20 suction release indentations formed within raised oblong shaped rings
21 raised ridge members
22 first continuous ridge member
23 second continuous ridge member
24 lip member
26 interior spiral ribbing
28 plurality of small openings

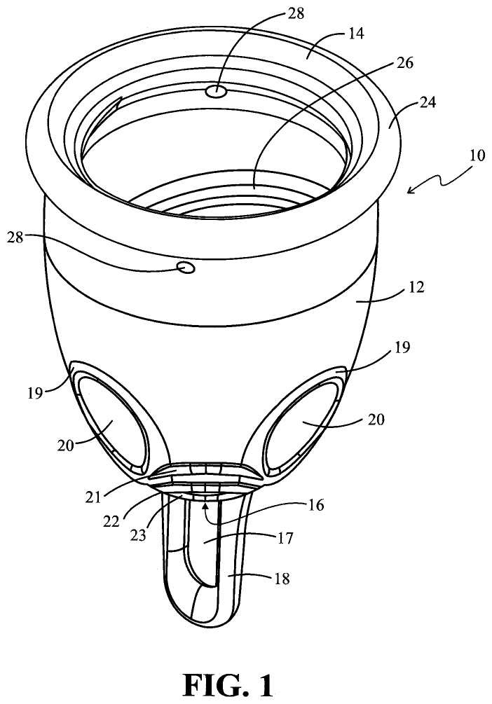

The exemplary embodiments of the menstrual cup of the present invention provide a user with a durable and reliable menstrual cup that protects from leakage around the rim and that enables swift and easy removal of the cup from the inside of the user's body. Although FIGS. 1-5 include numerous features and elements that are all included in one exemplary embodiment of the invention, other exemplary embodiments of the menstrual cup of the present invention may include fewer features and elements that those shown in FIGS. 1-5 while still enabling the menstrual cup to exhibit its improved functions of protecting from leakage and enabling swift and easy removal by a user.

FIG. 1 is a perspective view of an exemplary embodiment of the menstrual cup 10 of the present invention which includes a cup shaped main body 12 having an open top 14 and a closed bottom 16 and an elongated loop member 18 attached to an exterior of the closed bottom 16 of the main body 12. The elongated loop member 18 has an opening 17 through which a user may insert one or more fingers to assist in removing the menstrual cup 10 from inside of the user's body. Menstrual cup 10 also includes a plurality of raised oblong shaped rings 19 equidistantly spaced apart from one another on an exterior surface of the cup shaped main body 12 near the closed bottom 16 of the main body 12 which in turn form a plurality of suction release indentations 20. A plurality of raised ridge members 21 are positioned on the exterior surface of the cup shaped main body 12 such that each raised exterior ridge member 21 connects to two of the raised oblong shaped rings 19 thereby connecting all of the raised oblong shaped rings 19 to one another. In addition, a first continuous ridge member 22 extends about an outer circumference of the cup shaped main body 12 on the exterior surface of the main body 12 between the plurality of raised ridge members 21 and the closed bottom 16 of the main body 12, and a second continuous ridge member 23 extends about an outer circumference of the cup shaped main body 12 on the exterior surface of the main body 12 between the first continuous ridge member 22 and the closed bottom 16 of the main body 12 such that it touches opposing sides of the elongated loop member 18. Menstrual cup 10 also includes a lip member 24 positioned about an outer circumference of the open top 14 of the cup shaped main body 12 and an interior spiral ribbing 26 located about an interior surface of the cup shaped main body 12 extending from the open top 14 of the main body 12 to the closed bottom 16 of the main body 12. Menstrual cup 10 also includes a plurality of small openings 28 extending through the cup shaped main body 12 located near the open top 14 of the main body 12 and spaced equidistantly from one another about a circumference of the main body 12. Although the plurality of small openings 28 are spaced about a circumference of the cup shaped main body 12 near the open top 14 of the main body 12 and the plurality of raised oblong shaped rings 19 are positioned about a circumference on the exterior surface of the cup shaped main body 12 near the closed bottom 16 of the main body 12, the plurality of small openings 28 are spaced such that they are positioned between, and not directly above, the plurality of raised oblong shaped rings 19. As shown in FIGS. 1-4, the plurality of small openings 28 may include four small openings and the plurality of raised oblong shaped rings 19 may include four raised oblong shaped rings where the four small openings 28 are equidistantly spaced about the circumference of the cup shaped main body 12 near the open top 14 of the main body such that they are each located between, and not directly above, the four raised oblong shaped rings 19 that are equidistantly spaced apart from one another on the exterior surface of the cup shaped main member 12 bear the closed bottom 16 of the main body 12.

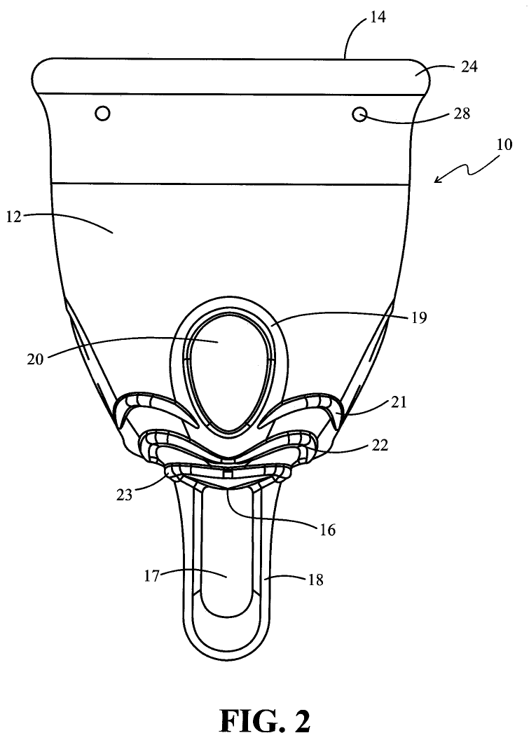

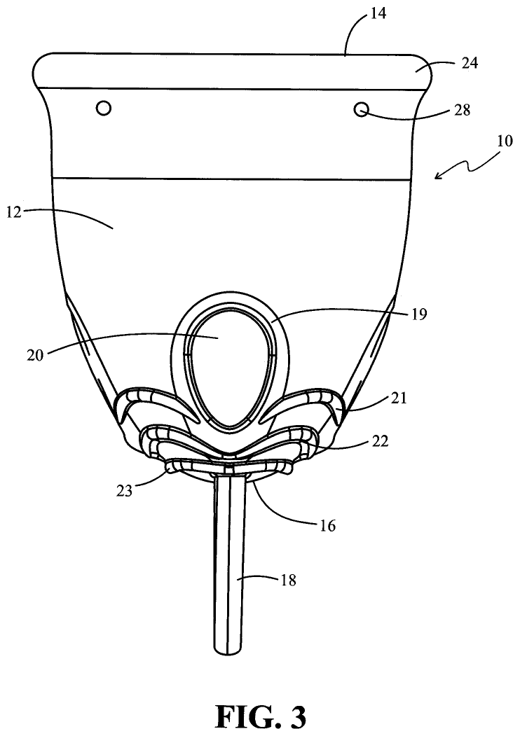

FIG. 2 is a front elevational view of the exemplary embodiment of the menstrual cup 10 shown in FIG. 1 and FIG. 3 is a side elevational view of the exemplary embodiment of the menstrual cup 10 shown in FIG. 1. The elongated loop member 18, the plurality of raised oblong shaped rings 19 forming suction release indentations 20, the plurality of small openings 28, and the interior spiral ribbing 26 each alone, and together in one or more combinations, assist a user in performing the swift and easy removal of the menstrual cup 10 from inside of a user. Second continuous ridge member 23, first continuous ridge member 22, and the plurality of raised ridge members 21 all provide additional structural strength to the lower part of cup shaped main body 12 to ensure it retains its cup like shape for retaining fluid and they also all provide assistance in guiding a user to the location of the suction release indentations 20 formed by the raised oblong shaped rings 19 to enable a user to swiftly and easily remove the menstrual cup 10 from inside of the user's body.

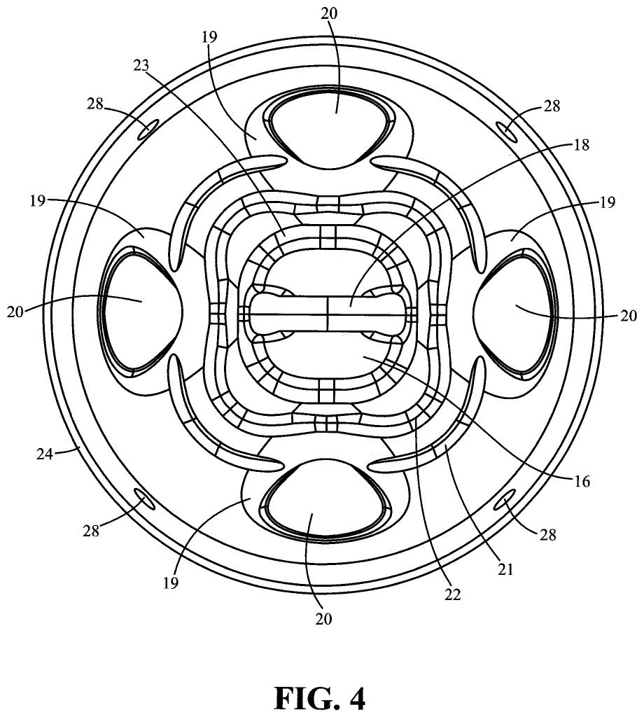

A bottom plan view of the exemplary embodiment of the menstrual cup 10 shown in FIG. 1 is depicted in FIG. 4. As shown in FIG. 4, the four small openings 28 are equidistantly spaced about the circumference of the cup shaped main body 12 near the open top 14 of the main body such that they are each located between, and not directly above, the four raised oblong shaped rings 19 that are equidistantly spaced apart from one another on the exterior surface of the cup shaped main member 12 bear the closed bottom 16 of the main body 12. In addition. FIG. 4 shows that each raised exterior ridge member 21 connects to two of the raised oblong shaped rings 19 thereby connecting all of the raised oblong shaped rings 19 to one another. In addition, FIG. 4 shows that first continuous ridge member 22 extends about an outer circumference of the cup shaped main body 12 on the exterior surface of the main body 12 between the plurality of raised ridge members 21 and the closed bottom 16 of the main body 12, and that second continuous ridge member 23 extends about an outer circumference of the cup shaped main body 12 on the exterior surface of the main body 12 between the first continuous ridge member 22 and the closed bottom 16 of the main body 12 such that it touches opposing sides of the elongated loop member 18.

FIG. 5 is a top plan view of the interior of the exemplary embodiment of the menstrual cup 10 shown in FIG. 1. As can be seen in FIG. 5, lip member 24 is positioned about an outer circumference of the open top 14 of the cup shaped main body 12 and interior spiral ribbing 26 is located about an interior surface of the cup shaped main body 12 extending from the open top 14 of the main body 12 to the closed bottom 16 of the main body 12.

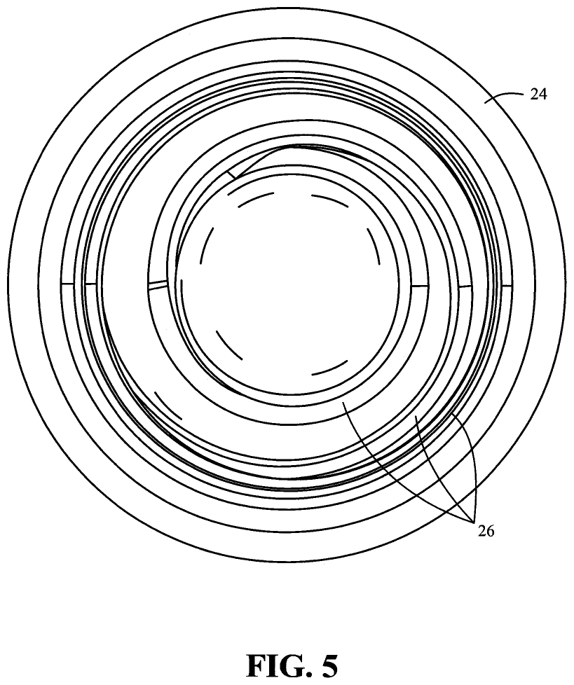

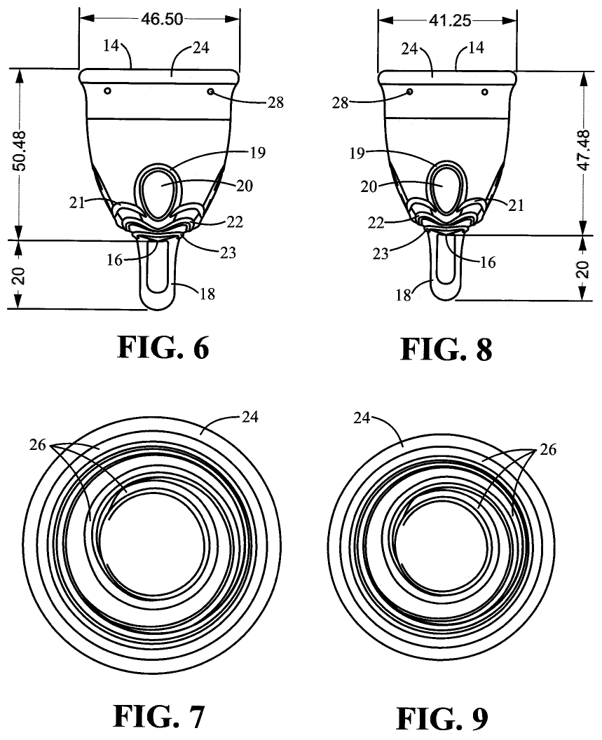

FIG. 6 is a front view showing dimensions for one size of the exemplary embodiment of the menstrual cup 10 shown in FIG. 1 and FIG. 7 is a top plan view showing the interior of the menstrual cup 10 shown in FIG. 6. The radius of the open top 14 of the cup shaped main body 12 extending from the lip member 24 on one side of the main body 12 to the lip member 24 on the other side of the main body 12 of the menstrual cup 10 shown in FIGS. 6 and 7 is about 46.5 millimeters and the height of the menstrual cup 10 from the open top 14 of the cup shaped main body to the closed bottom 16 of the cup shaped main body 12 is about 50.48 millimeters. The height of the elongated loop member is about 20 millimeters while the radius of each of the plurality of small openings 28 is about 1.0 to 1.5 millimeters.

FIG. 8 is a front view showing dimensions for one size of the exemplary embodiment of the menstrual cup 10 shown in FIG. 1 and FIG. 9 is a top plan view showing the interior of the menstrual cup 10 shown in FIG. 8. The radius of the open top 14 of the cup shaped main body 12 extending from the lip member 24 on one side of the main body 12 to the lip member 24 on the other side of the main body 12 of the menstrual cup 10 shown in FIGS. 6 and 7 is about 41.25 millimeters and the height of the menstrual cup 10 from the open top 14 of the cup shaped main body to the closed bottom 16 of the cup shaped main body 12 is about 47.48 millimeters. The height of the elongated loop member is about 20 millimeters while the radius of each of the plurality of small openings 28 is about 1.0 to 1.5 millimeters.

All exemplary embodiments of the menstrual cup 10 may comprise a soft flexible polymer material that can return to its original shape after being manipulated such as by squeezing or stretching the cup. Further, it will be understood by those skilled in the art that the exemplary embodiments of the menstrual cup 10 of the present invention described above and set forth in the claims may preferably be formed from one continuously molded piece.

The detailed description of exemplary embodiments of the invention herein show various exemplary embodiments and the best modes, known to the inventor at this time, of the invention. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those reasonably skilled in the art. Additionally, all included figures are non-limiting illustrations of the exemplary embodiments and modes, which similarly avail themselves to any equivalent modes or embodiments that are known or obvious to those reasonably skilled in the art.

Other combinations and/or modifications of structures, arrangements, applications, proportions, elements, materials, or components used in the practice of the instant invention, in addition to those not specifically recited, can be varied or otherwise particularly adapted to specific environments, manufacturing specifications, design parameters, or other operating requirements without departing from the scope of the instant invention and are intended to be included in this disclosure.

Unless specifically noted, it is the Applicant's intent that the words and phrases in the specification and the claims be given the commonly accepted generic meaning or an ordinary and accustomed meaning used by those of ordinary skill in the applicable arts. In the instance where these meanings differ, the words and phrases in the specification and the claims should be given the broadest possible, generic meaning. If any other special meaning is intended for any word or phrase, the specification will clearly state and define the special meaning.

1. A menstrual cup comprising:
a cup shaped main body having an open top and a closed bottom;
a spiral ribbing located about an interior surface of the cup shaped main body extending from the open top of the main body to the closed bottom of the main body;
a plurality of openings extending through the cup shaped main body located near the open top of the main body and spaced equidistantly from one another about a circumference of the main body; and
an elongated loop member attached to an exterior of the closed bottom of the main body.

2. The menstrual cup of claim 1 further comprising a lip member positioned about an outer circumference of the open top of the cup shaped main body.

3. The menstrual cup of claim 1 wherein the plurality of openings extending through the cup shaped main body comprise four openings.

4. The menstrual cup of claim 1 further comprising a plurality of raised oblong shaped rings equidistantly spaced apart from one another on an exterior surface of the cup shaped main body near the closed bottom of the main body.

5. The menstrual cup of claim 4 wherein the plurality of raised oblong shaped rings comprise four raised oblong shaped rings.

6. The menstrual cup of claim 5 further comprising a plurality of raised ridge members positioned on the exterior surface of the cup shaped main body such that each raised ridge member connects to two of the raised oblong shaped rings.

7. The menstrual cup of claim 6 further comprising a first continuous ridge member that extends about an outer circumference of the cup shaped main body on the exterior surface of the main body between the plurality of raised ridge members and the closed bottom of the main body.

8. The menstrual cup of claim 7 further comprising a second continuous ridge member that extends about an outer circumference of the cup shaped main body on the exterior surface of the main body between the first continuous ridge member and the closed bottom of the main body such that it touches opposing sides of the elongated loop member.

9. A menstrual cup comprising:
a cup shaped main body having an open top and a closed bottom;
a plurality of raised oblong shaped rings equidistantly spaced apart from one another on an exterior surface of the cup shaped main body near the closed bottom of the main body;
a plurality of raised exterior ridge members positioned on the exterior surface of the cup shaped main body such that each raised exterior ridge member connects to two of the raised oblong shaped rings thereby connecting all of the raised oblong shaped rings to one another; and
an elongated loop member attached to an exterior of the closed bottom of the main body.

10. The menstrual cup of claim 9 further comprising a spiral ribbing located about an interior surface of the cup shaped main body extending from the open top of the man body to the closed bottom of the main body.

11. The menstrual cup of claim 9 wherein the plurality of raised oblong shaped rings comprise four raised oblong shaped rings.

12. The menstrual cup of claim 9 further comprising a plurality of openings extending through the cup shaped main body located near the open top of the main body and spaced equidistantly from one another about a circumference of the main body.

13. The menstrual cup of claim 12 wherein the plurality of openings extending through the cup shaped main body comprise four openings.

14. The menstrual cup of claim 9 further comprising a lip member positioned about an outer circumference of the open top of the cup shaped main body.

15. The menstrual cup of claim 9 further comprising a first continuous ridge member that extends about an outer circumference of the cup shaped main body on the exterior surface of the main body between the plurality of raised ridge members and the closed bottom of the main body.

16. The menstrual cup of claim 15 further comprising a second continuous ridge member that extends about an outer circumference of the cup shaped main body on the exterior surface of the main body between the first continuous ridge member and the closed bottom of the main body such that it touches opposing sides of the elongated loop member.

17. A menstrual cup comprising:
a cup shaped main body having an open top and a closed bottom;
a spiral ribbing located about an exterior surface of the cup shaped main body extending from the open top of the main body to the closed bottom of the main body, a plurality of openings extending through the cup shaped main body located near the open top of the main body and spaced equidistantly from one another about a circumference of the main body;
a plurality of raised oblong shaped rings equidistantly spaced apart from one another on an exterior surface of the cup shaped main body near the closed bottom of the main body; and
an elongated loop member attached to an exterior of the closed bottom of the main body.

18. The menstrual cup of claim 17 wherein the plurality of openings extending through the cup shaped main body comprise four openings.

19. The menstrual cup of claim 17 wherein the plurality of raised oblong shaped rings comprise four raised oblong shaped rings.

20. The menstrual cup of claim 17 wherein the cup shaped main body, the spiral ribbing, the plurality of openings, the plurality of raised oblong shaped rings, and the elongated loop member are all formed from one molded piece.
